(19) United States
(12) Patent Application Publication
(10) Pub. No.: US 2017/0275568 A1
(43) Pub. Date: Sep. 28, 2017

(54) DETERGENT OR CLEANING AGENT WITH SPECIAL a-AMYLASE AND DEFINED WATER ACTIVITY Aw

(71) Applicant: Henkel AG & Co. KGaA, Duesseldorf (DE)

(72) Inventors: Marouane Antir, Hilversum (NL); Sabine Schuemann, Neuss (DE); Thorsten Bastigkeit, Wuppertal (DE); Martina Seiler, Duisburg (DE); Timothy O'Connell, Landsberg am Lech (DE); Frank Meier, Duesseldorf (DE); Dirk Weinrich, Grevenbroich (DE); Leyla Koelling, Hilden (DE); Luca Bellomi, Dusseldorf (DE); Sheila Edwards, Duesseldorf (DE)

(73) Assignee: Henkel AG & Co. KGaA, Duesseldorf (DE)

(21) Appl. No.: 15/616,994

(22) Filed: Jun. 8, 2017

Related U.S. Application Data
(63) Continuation of application No. PCT/EP2015/075798, filed on Nov. 5, 2015.

(30) Foreign Application Priority Data
Dec. 10, 2014 (DE) 10 2014 225 478.7

(51) Int. Cl. C11D 3/386; C12N 15/00; C12N 1/38; C12N 9/28

(52) U.S. Cl. CPC C11D 3/386; C12N 9/2417; C12N 15/00; C12N 1/38; C12N 9/00

(57) ABSTRACT

The invention relates to liquid compositions, in particular for cleaning textiles, containing: (a) at least one surfactant, and (b) at least one α-amylase, which is at least 89% and increasing preferably at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and up to 100% identical to the sequence set forth in SEQ ID NO. 1 over the entire length thereof and has deletions at one or more of positions 180, 181, 182, 183, and 184 in the counting according to SEQ ID NO. 1, with the stipulation that the composition has a water activity (a_{w}) between 0.3 and 0.95, in particular in a range of 0.4 to 0.95, at 25° C. and 1013 mbar, stabilize in particular the amylase.

### FIELD OF THE INVENTION

The present invention generally relates to the cleaning of surfaces, particularly of textiles, as well as to the provision of liquid compositions for this purpose.

### BACKGROUND OF THE INVENTION

Usually, one and the same detergent and cleaning agent is used to remove a wide variety of contaminants on substrates. In order to meet demands for the greatest possible effective removal of stains, detergents and cleaning agents contain a wide variety of active ingredients. A person skilled in the art is familiar with contaminants, for example, that can be better removed through the use of enzymes—so-called enzyme-sensitive contaminants. Therefore, in addition to the surfactants that are commonly used for soil removal of enzyme-sensitive contaminants, the enzymes protease, lipase, amylase, and mannanase, for example, are additionally added to detergents or cleaning agents.

In order to remove starch-based contaminants, detergents or cleaning agents often contain the enzyme α-amylase. Synonymous terms—such as 1,4-alpha-D-glucan glucanohydrolase or glycogenase, for example—can be used for amylases. Preferred amylases are oftentimes α-amylases. The crucial factor for determining whether an enzyme is an α-amylase in terms of the invention is its ability to hydrolyze α-(1,4) glycoside bonds in the amylose of the starch.

Amylases known from the prior art, particularly including improved developments thereof for use in detergents or cleaning agents, will be outlined below. The α-amylase from Bacillus licheniformis (available as Termamyl® from Novozymes or Purastar®ST from Danisco/Genencor) and further-developed products Duramyl® and Termamyl®ultra (Novozymes), Purastar®OxAm (Danisco/Genencor), and Keistase® (Daiwa Seiko lnc.), as well as the α-amylases from Bacillus amyloliquefaciens or from Bacillus stearothermophilus, are suitable amylases. The α-amylase from Bacillus amyloliquefaciens is sold by Novozymes under the name BAN®, and variants derived from the α-amylase from Bacillus stearothermophilus are sold under the names BSG® and Novamyl®, also by Novozymes. Other known amylases are the α-amylase from Bacillus sp. A 7-7 (DSM 12368) and the cyclodextrin glucanotransferase (CGTase) from Bacillus agaradherens (DSM 9948), as well as the further developments of the α-amylase from Aspergillus niger and A. oryzae available from Novozymes under the trade name Fungamyl®. Examples of other commercial products that can be advantageously used in detergents and cleaning agents are Amylase-LT® and Stainzyme® or Stainzyme ultra® and Stainzyme plus®, the latter also being available from Novozymes. Variants of these enzymes that can be obtained through point mutation can also be used in detergents and cleaning agents. Especially prominent amylases are disclosed in international published patent applications WO 0060060, WO 03002711, WO 03054177, and WO 07079938.

The activity of the amylase during the washing or cleaning process depends on various factors. Besides the temperature and the pH value of the detergent or cleaning agent, the storage stability of the enzyme in the detergent or cleaning agent is a crucial factor. A high degree of enzyme activity should also be ensured for the entire period of time from the production of the detergent or cleaning agent through the last use by the consumer.

Accordingly, it is desirable to provide liquid compositions for washing or cleaning compositions for textiles that have outstanding washing performance with regard to the removal of starch-based contaminants. Preferably, these effects are to be achieved at application temperatures that are as low as possible, particularly between 10° C. and 40° C. In addition, it is desirable that the effects of the liquid compositions are contributed in part by enzymes contained therein. Furthermore, it is desirable that the washing performance provided by the enzyme should be sustained to the greatest possible extent over the storage period of the detergent or cleaning agent.

Other desirable features and characteristics of the present invention will become apparent from the subsequent detailed description of the invention and the appended claims, taken in conjunction with this background of the invention.

### BRIEF SUMMARY OF THE INVENTION

A first object of the invention is a liquid composition, particularly for laundry or for cleaning textiles, containing
(a) at least one surfactant, and
(b) at least one α-amylase that is at least 89% and, in order of increasing preference, at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and up to 100% identical to the sequence indicated in SEQ ID NO.1 over its entire length and, in the count according to SEQ ID NO. 1, has deletions at one or more of positions 180, 181, 182, 183, and 184,
   with the proviso that the composition has a water activity a_{w} between 0.3 and 0.95 at 25° C. and 1013 mbar.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

In terms of the invention, and in keeping with general linguistic use, when a numerical range is defined as lying “between” two range limits, the range limits are not included. Numerical ranges that are defined by a range limit up to another range limit include the range limits.

The composition according to the invention is liquid at 25° C. and 1013 mbar.

For their cleaning or washing performance, the compositions according to the invention contain at least one surfactant, especially preferably a mixture of several surfactants from different substance classes.

It is preferred for the invention if the total quantity of surfactant is 2.0 to 70 wt %, preferably 5.0 to 60.0 wt %, more preferably from 10.0 to 40.0 wt %, and especially preferably from 15.0 to 36.0 wt %, each with respect to the total weight of the composition.

It is preferred according to the invention if at least one anionic surfactant is used as a surfactant.

All anionic surface-active substances are suitable for use as anionic surfactants in the compositions. These are characterized by a water-solubilizing, anionic group such as a carboxylate, sulfate, sulfonate or phosphate group and a lipophilic alkyl group with about 8 to 30 C atoms. In addition, glycol or polyglycol ether groups, ester, ether and amide groups as well as hydroxyl groups can be contained in the molecule. Suitable anionic surfactants are preferably present in the form of the sodium, potassium and ammonium as well as the mono-, di- and trialkanol ammonium salts with 2 to 4 C atoms in the alkanol group.

With respect to the total weight of the composition, preferred compositions contain anionic surfactant in a total quantity from 1.0 to 35.0 wt %, preferably from 5.0 to 30.0 wt %, and especially preferably from 10.0 to 25.0 wt %.

Preferred anionic surfactants in the compositions are alkyl sulfates, alkyl polyglycol ether sulfates, and ether carboxylic acids, each with 10 to 18 C atoms in the alkyl group and up to 12 glycol ether groups in the molecule.

Compositions that are preferably used according to the invention contain at least one surfactant of the formula

R¹—O-(AO)ₙ—SO₃⁻ X⁺.

In this formula, R¹ stands for a linear or branched, substituted or unsubstituted alkyl, aryl, or alkylaryl residue, preferably for a linear, unsubstituted alkyl residue, especially preferably for a fatty alcohol residue. Preferred residues R¹ are selected from among decyl, undecyl, dodecyl, tridecyl, tetradecyl, pentadecyl, hexadecyl, heptadecyl, octadecyl, nonadecyl, and eicosyl residues and mixtures thereof, with the representatives with an even number of C atoms being preferred. Especially preferred residues R¹ are derived from C₁₂-C₁₈ fatty alcohols, for example from coconut fatty alcohol, tallow fatty alcohol, lauryl, myristyl, cetyl, or stearyl alcohol, or from C₁₀-C₂₀ oxo alcohols.

AO stands for an ethylene oxide (EO) or propylene oxide (PO) group, preferably for an ethylene oxide group. The index n stands for an integer from 1 to 50, preferably from 1 to 20, and particularly from 2 to 10. Very especially preferably, n stands for the numbers 2, 3, 4, 5, 6, 7, or 8. X stands for a monovalent cation or the nth part of an n-valent cation, with the alkali metal ions and, among those, Na⁺ or K⁺, being preferred, and with Na⁺ being most preferred. Other cations X⁺ can be selected from among NH₄⁺, ½ Zn₂⁺, ½ Mg₂⁺, ½ Ca₂⁺, ½ Mn₂⁺, and mixtures thereof.

In summary, especially preferred compositions contain at least one anionic surfactant selected from among fatty alcohol ether sulfates of formula A-1

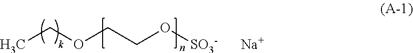

where k=11 to 19, n=2, 3, 4, 5, 6, 7, or 8. Very especially preferred representatives are Na—C₁₂₋₁₄ fatty alcohol ether sulfates with 2 EO (k=11-13, n=2 in formula A-1).

Preferred compositions contain 1.0 to 15 wt %, preferably 2.5 to 12.5 wt %, more preferably 5.0 to 10.0 wt % fatty alcohol ether sulfate(s) with respect to the total quantity of the composition (each particularly of formula A-1).

Other preferred composition contain, in addition or alternatively (particularly in addition), at least one surfactant of the formula (A-2)

R³-A-SO₃⁻ Y⁺ (A-2).

In this formula, R³ stands for a linear or branched, substituted or unsubstituted alkyl, aryl, or alkylaryl residue, and the group -A- stands for —O— or a chemical bond. In other words, sulfate surfactants (A=O) or sulfonate surfactants (A=chemical bond) can be described by the above formula. Certain residues R³ are preferred depending on the choice of group A. In the sulfate surfactants (A=O), R³ preferably stands for a linear, unsubstituted alkyl residue, especially preferably for a fatty alcohol residue. Preferred residues R¹ are selected from among decyl, undecyl, dodecyl, tridecyl, tetradecyl, pentadecyl, hexadecyl, heptadecyl, octadecyl, nonadecyl, and eicosyl residues and mixtures thereof, with the representatives with an even number of C atoms being preferred. Especially preferred residues R¹ are derived from C₁₂-C₁₈ fatty alcohols, for example from coconut fatty alcohol, tallow fatty alcohol, lauryl, myristyl, cetyl, or stearyl alcohol, or from C₁₀-C₂₀ oxo alcohols. Y stands for a monovalent cation or the nth part of an n-valent cation, with the alkali metal ions and, among those, Na+ or K+, being preferred, and with Na+ being most preferred. Other cations Y⁺ can be selected from among NH₄⁺, ½ Zn₂⁺, ½ Mg₂⁺, ½ Ca₂⁺, ½ Mn₂⁺, and mixtures thereof.

Such especially preferred surfactants are selected from among fatty alcohol sulfate of formula A-2a

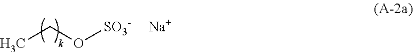

where k=11 to 19. Very especially preferred representatives are Na—C₁₂₋₁₄ fatty alcohol sulfates (k=11-13 in formula A-2a).

Other preferred compositions contain, with respect to the total quantity of the compositions, 1.0 to 25.0 wt %, preferably 2.5 to 20.5 wt %, more preferably 5.0 to 20.0 wt %, surfactant from the group comprising C₉₋₁₃ alkylbenzene sulfonates, olefin sulfonates, C₁₂₋₁₈ alkane sulfonates, ester sulfonates, alk(en)yl sulfates, and mixtures thereof (particularly from the group of the C₉₋₁₃ alkylbenzene sulfonates, preferably from the group according to formula (A2)).

In the sulfate surfactants (A=chemical bond) that are preferred over the sulfate surfactants of the above formula, R³ preferably stands for a linear or branched unsubstituted alkylaryl residue. Here, too, X stands for a monovalent cation or the nth part of an n-valent cation, with the alkali metal ions and, among those, Na+ or K+, being preferred, and with Na+ being most preferred. Other cations X⁺ can be selected from among NH₄⁺, ½ Zn₂⁺, ½ Mg₂⁺, ½ Ca₂⁺, ½ Mn₂⁺, and mixtures thereof.

Such most preferred surfactants are selected from among linear or branched alkylbenzene sulfonates of formula A-3

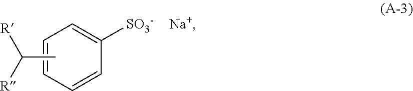

in which R′ and R″ together contain 9 to 19, preferably 11 to 15, and particularly 11 to 13 C atoms. One very especially preferred representative can be described by formula A-3a:

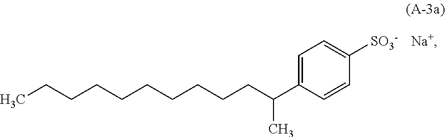

It has proven expedient for cold washing performance if the compositions contain additional soap(s) as anionic surfactant. Soaps are the water-soluble sodium or potassium salts of the saturated and unsaturated fatty acids with 10 to 20 carbon atoms, of the resin acids of rosin (yellow resin soaps) and of the naphthenic acids that are used as solid or semisolid mixtures primarily for purposes of washing and cleaning. Sodium or potassium salts of the saturated and unsaturated fatty acids with 10 to 20 carbon atoms, particularly with 12 to 18 carbon atoms, are preferred soaps according to the invention. Especially preferred compositions are characterized in that they contain 0.1 to 6 wt %, especially preferably 0.2 to 4.5 wt %, very especially preferably 0.3 to 4.1 wt % soap(s), with respect to their weight.

To better achieve the technical object, it is very especially preferred according to the invention to use a combination of
at least one fatty alcohol ether sulfate of formula A1

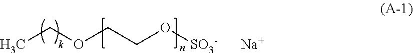

where k=11 to 19, n=2, 3, 4, 5, 6, 7, or 8 (especially preferred representatives are Na—C₁₂₋₁₄ fatty alcohol ether sulfates with 2 EO (k=11-13, n=2 in formula A-1), and
at least one linear or branched alkylbenzene sulfonate of formula A-3

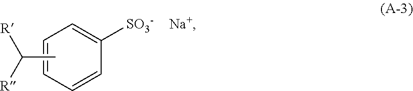

in which R′ and R″ together contain 9 to 19, preferably 11 to 15, and particularly 11 to 13 C atoms (particularly of the above formula (A-3a)),
in the compositions according to the invention. Preferred compositions of this embodiment are optionally preferably characterized in that they contain one or more soaps (preferably, with respect to their weight, 0.1 to 6 wt %, especially preferably 0.2 to 4.5 wt %, very especially preferably 0.3 to 4.1 wt % soap(s)).

In addition to the anionic surfactant(s), or in place of them, the compositions used according to the invention can contain nonionic surfactant(s).

Especially preferably, the compositions contain at least one nonionic surfactant from the group of the fatty alcohol ethoxylates, since these surfactants result in powerful compositions even at low washing temperatures and, in the case of liquid preparations, have excellent low-temperature stability.

Accordingly, preferred compositions additionally contain at least one nonionic surfactant of the formula

R²—O-(AO)ₙ—H,

in which
R² stands for a linear or branched, substituted or unsubstituted alkyl, aryl, or alkylaryl residue,
AO stands for an ethylene oxide (EO) or propylene oxide (PO) group,
m stands for integers from 1 to 50.

In the abovementioned formula, R² stands for a linear or branched, substituted or unsubstituted alkyl, aryl, or alkylaryl residue, preferably for a linear, unsubstituted alkyl residue, especially preferably for a fatty alcohol residue. Preferred residues R² are selected from among decyl, undecyl, dodecyl, tridecyl, tetradecyl, pentadecyl, hexadecyl, heptadecyl, octadecyl, nonadecyl, and eicosyl residues and mixtures thereof, with the representatives with an even number of C atoms being preferred. Especially preferred residues R² are derived from C₁₂-C₁₈ fatty alcohols, for example from coconut fatty alcohol, tallow fatty alcohol, lauryl, myristyl, cetyl, or stearyl alcohol, or from C₁₀-C₂₀ oxo alcohols.

AO stands for an ethylene oxide (EO) or propylene oxide (PO) group, preferably for an ethylene oxide group. The index m stands for an integer from 1 to 50, preferably from 1 to 20, and particularly from 2 to 10. Very especially preferably, m stands for the numbers 2, 3, 4, 5, 6, 7, or 8.

In summary, especially preferred surfactants are selected from among fatty alcohol ethoxylates of formula C-1

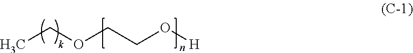

where k=11 to 19, m=2, 3, 4, 5, 6, 7, or 8. Very especially preferred representatives are C₁₂₋₁₄ fatty alcohols with 7 EO (k=11-17, m=7 in formula C-1).

Particularly preferred compositions contain nonionic surfactants in certain quantities. The most preferred compositions according to the invention are characterized in that the total quantity of nonionic surfactants, with respect to the weight of the compositions, is 1.0 to 25 wt %, preferably 2.5 to 20.0 wt %, more preferably 5.0 to 18.0 wt %.

Other preferred compositions contain 1.0 to 25 wt %, preferably 2.5 to 20.0 wt %, more preferably 5.0 to 18.0 wt % fatty alcohol ethoxylate(s) with respect to the total quantity of the compositions (each particularly of formula C-1).

Preferred compositions contain, with respect to the total weight of the composition,
anionic surfactant in a total quantity from 1.0 to 35.0 wt %, preferably from 5.0 to 30.0 wt %, and especially preferably from 10.0 to 25.0%, and
nonionic surfactant in a total quantity from 1.0 to 25 wt %, preferably 2.5 to 20.0 wt %, and more preferably from 5.0 to 18.0%.

Here again, it is preferred that the preferred surfactants be used (most preferably in the quantities characterized as being preferred). The quantity of the special anionic and nonionic surfactants is to be selected in this case such that the previously defined total quantity of anionic surfactant and the previously defined total quantity of nonionic surfactant is observed.

According to an especially preferred embodiment, such compositions are preferred which contain
i) at least one anionic surfactant of the formula R¹—O-(AO)ₙ—SO₃⁻ X⁺, and
ii) at least one anionic surfactant of formula A-3

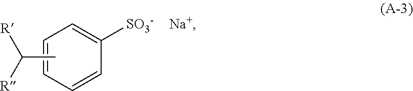

and
iii) at least one anionic surfactant of the formula R²—O-(AO)ₘ—H, in which
R¹ stands for a linear or branched, substituted or unsubstituted alkyl, aryl, or alkylaryl residue,
R′ and R″ together contain 9 to 19, preferably 11 to 15, and particularly 11 to 13 C atoms,
AO stands, independently of one another, for an ethylene oxide (EO) or propylene oxide (PO) group,
n, m stand, independently of one another, for integers from 1 to 50,
X stands for a monovalent cation or the nth part of an n-valent cation.

The surfactants i) and ii) were described above as preferred surfactants a) with the formulas (A-1) and (A-3a), and the surfactant iii) was described as a preferred surfactant with the formula (C-1). Preferred compositions of this embodiment are, in turn, characterized in that they additionally contain at least one soap.

According to a very especially preferred embodiment, such compositions are preferred which contain, with respect to the weight of the composition,
i) at least one anionic surfactant of the formula R¹—O-(AO)ₙ—SO₃— X⁺ in a total quantity from 1.0 to 15 wt %, preferably 2.5 to 12.5 wt %, more preferably 5.0 to 10.0 wt %, and
ii) at least one anionic surfactant of formula A-3 in a total quantity from 1.0 to 25 wt %, preferably 2.5 to 20.5 wt %, more preferably 5.0 to 20.0 wt %,

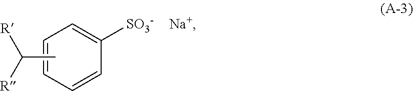

and
iii) at least nonionic surfactant of the formula R²—O-(AO)ₘ—H in a total quantity from 1.0 to 25 wt %, preferably 2.5 to 20.0 wt %, more preferably 5.0 to 18.0 wt %,
   in which
R¹ stands for a linear or branched, substituted or unsubstituted alkyl, aryl, or alkylaryl residue,
R′ and R″ together contain 9 to 19, preferably 11 to 15, and particularly 11 to 13 C atoms,
AO stands, independently of one another, for an ethylene oxide (EO) or propylene oxide (PO) group,
n, m stand, independently of one another, for integers from 1 to 50,
X stands for a monovalent cation or the nth part of an n-valent cation.

Especially preferred compositions of this embodiment are, in turn, characterized in that they contain 0.1 to 6 wt %, especially preferably 0.2 to 4.5 wt %, very especially preferably 0.3 to 4.1 wt % soap(s) with respect to their weight.

The composition according to the invention can contain water as a solvent. Compositions that are preferred according to the invention contain water in a quantity between 0 to 55 wt %, particularly from 2 to 50 wt %, each with respect to the total weight of the composition.

It is preferred in this regard that the detergent contain greater than 5 wt %, preferably greater than 15 wt %, and particularly preferably greater than 25 wt %, very especially preferably greater than 40 wt % water, each with respect to the total quantity of detergent. It is also preferred in this regard that the detergent contain less than 55 wt %, preferably less than 50 wt %, and particularly preferably less than 45 wt %, each with respect to the total quantity of detergent.

Alternatively and especially preferably, the detergents have a low water content, with the water content in one preferred embodiment being less than 35 wt %, more preferably less than 30 wt %, especially preferably less than 10 wt %, very especially preferably less than 8 wt %, each with respect to the overall liquid detergent.

The thermoforming process generally includes the forming of a first layer of a water-soluble film material in order to form at least one recess for receiving at least one respective composition therein, filling of the composition into the respective recess, covering of the recesses filled with the composition with a second layer of a water-soluble film material, and sealing of the first and second layers with one another at least around the recesses.

The water-soluble film preferably contains at least one water-soluble polymer as a film material. The covering for the liquid composition according to the invention can be formed from one or two or more layers of a water-soluble film material. The water-soluble film material of the first layer and of the additional layers, if existent, can be the same or different.

It is preferred that the water-soluble film material contain polyvinyl alcohol or a polyvinyl alcohol copolymer.

Suitable water-soluble films are preferably based on a polyvinyl alcohol or a polyvinyl alcohol copolymer whose molecular weight lies in the range from 10,000 to 1,000,000 gmol⁻¹, preferably from 20,000 to 500,000 gmol⁻¹, especially preferably from 30,000 to 100,000 gmol⁻¹, and particularly from 40,000 to 80,000 gmol⁻¹.

The manufacture of polyvinyl alcohol is usually done through the hydrolysis of polyvinyl acetate, since the direct synthetic pathway is not possible. The same applies to polyvinyl alcohol copolymers, which are manufactured analogously from polyvinyl acetate copolymers. It is preferred if at least one layer of the water-soluble material comprises a polyvinyl alcohol whose degree of hydrolysis makes up 70 to 100 mol %, preferably 80 to 90 mol %, especially preferably 81 to 89 mol %, and particularly 82 to 88 mol %.

Polymers selected from the group comprising acrylic acid-containing polymers, polyacrylamides, oxazoline polymers, polystyrene sulfonates, polyurethanes, polyesters, polyethers, polylactic acid, and/or mixtures of the above polymers can be additionally added to the water-soluble film material.

Besides vinyl alcohol, preferred polyvinyl alcohol copolymers comprise dicarboxylic acids as additional monomers. Suitable dicarboxylic acids are itaconic acid, malonic acid, succinic acid, and mixtures thereof, with itaconic acid being preferred.

Polyvinyl alcohol copolymers that are likewise preferred comprise, besides vinyl alcohol, an ethylenically unsaturated carboxylic acid, or a salt or ester thereof. Besides vinyl alcohol, such polyvinyl alcohol copolymers especially preferably contain acrylic acid, methacrylic acid, acrylic acid ester, methacrylic acid ester, or mixtures thereof.

Suitable water-soluble films for the manufacture of the water-soluble portion are films that are sold by MonoSol LLC under the name Monosol M8630. Other suitable films include films with the name Solublon® PT, Solublon® KA, Solublon® KC, or Solublon® KL from Aicello Chemical Europe GmbH or the films VF-HP from Kuraray, or HiSelon SH2312 from Nippon Gohsei.

Portions that are especially preferred according to the invention each contain in the liquid composition packaged therein according to the invention, with respect to the total weight of the liquid composition, a total quantity of
30 to 40 wt % of at least one anionic surfactant and
18 to 28 wt % of at least one nonionic surfactant.

Inventive liquid compositions that are very especially preferred according to the invention for packaging in said portion contain, as a surfactant, at least one of the preferred combinations (A) to (D):
(A) With respect to the total weight of the liquid composition, a respective total quantity of
   25 to 60 wt % of at least one anionic surfactant, with at least one C₉₋₁₃ alkylbenzene sulfonate being contained as an anionic surfactant, and
   2 to 35 wt % of at least one nonionic surfactant, with at least one alkoxylated alcohol with 8 to 18 carbon atoms and an average of 4 to 12 mols of ethylene oxide (EO) per mol of alcohol being contained as a nonionic surfactant.
(B) With respect to the total weight of the liquid composition, a respective total quantity of
   25 to 60 wt % of at least one anionic surfactant, with at least 25 to 60 wt % of at least one C₉₋₁₃ alkylbenzene sulfonate being contained as an anionic surfactant, and
   2 to 35 wt % of at least one nonionic surfactant, with at least 2 to 35 wt % of at least one alkoxylated alcohol with 8 to 18 carbon atoms and an average of 4 to 12 mols of ethylene oxide (EO) per mol of alcohol being contained as a nonionic surfactant.
(C) With respect to the total weight of the liquid composition, a respective total quantity of
   30 to 40 wt % of at least one anionic surfactant, with at least one C₉₋₁₃ alkylbenzene sulfonate being contained as an anionic surfactant, and
   18 to 28 wt % of at least one nonionic surfactant, with at least one alkoxylated alcohol with 8 to 18 carbon atoms and an average of 4 to 12 mols of ethylene oxide (EO) per mol of alcohol being contained as a nonionic surfactant.
(D) With respect to the total weight of the liquid composition, a respective total quantity of
   30 to 40 wt % of at least one anionic surfactant, with at least 15 to 30 wt % of at least one C₉₋₁₃ alkylbenzene sulfonate being contained as an anionic surfactant, and
   18 to 28 wt % of at least one nonionic surfactant, with at least 15 to 28 wt % of at least one alkoxylated alcohol with 8 to 18 carbon atoms and an average of 4 to 12 mols of ethylene oxide (EO) per mol of alcohol being contained as a nonionic surfactant.

Besides the components that must necessarily be present according to the invention, nonaqueous solvents can be added to the liquid composition according to the invention. Suitable nonaqueous solvents include mono- or polyvalent alcohols, alkanolamines, or glycol ethers, provided that they are miscible with water in the indicated concentration range. Preferably, the solvents are selected from among ethanol, n-propanol, i-propanol, butanols, glycol, propanediol, butanediol, methylpropanediol, glycerin, diglycol, propyl diglycol, butyl diglycol, hexylene glycol, ethylene glycol methyl ether, ethylene glycol ethyl ether, ethylene glycol propyl ether, ethylene glycol mono-n-butyl ether, diethylene glycol ethyl ether, diethylene glycol ethyl ether, propylene glycol methyl ether, propylene glycol ethyl ether, propylene glycol propyl ether, dipropylene glycol monomethyl ether, dipropylene glycol monoethyl ether, methoxytriglycol, ethoxytriglycol, butoxytriglycol, 1-butoxyethoxy-2-propanol, 3-methyl-3-methoxybutanol, propylene-glycol-t-butyl ether, di-n-octyl ether, as well as mixtures of these solvents. It is preferred, however, that the composition according to the invention contain an alcohol, particularly ethanol and/or glycerin and/or 1,2-propanediol, in quantities from 0.5 to 5 wt % with respect to the overall composition.

The liquid composition according to the invention must necessarily contain at least one special α-amylase as an enzyme.

The following terms and definitions also apply in the context of the present invention with regard to the definition of enzymes.

At the level of the proteins, “variant” is the term at the level of the nucleic acids that corresponds to “mutant.” The precursor or parent molecules can be wild-type enzymes, meaning those which can be obtained from natural sources. They can also be enzymes that already represent variants, that is, that have already been altered in relation to wild-type molecules. These are to be understood, for example, as being point mutants, those with modifications of the amino acid sequence, regions that are contiguous over several positions or more, or also hybrid molecules that are composed of mutually complementary sections of various wild-type enzymes.

As a matter of principle, the amino acid exchanges according to the invention named in the present invention are not limited to being the only exchanges in which the respective variant differs from the parent molecule. It is known from the prior art that the advantageous characteristics of individual point mutations can complement one another. Embodiments of the present invention therefore include all variants which, in addition to other exchanges in relation to the parent molecule, also have the exchanges according to the invention.

Furthermore, it is irrelevant in principle in what sequence the respective amino acid exchanges were performed, that is, whether a corresponding point mutant is developed according to the invention or a variant according to the invention is produced initially from a parent molecule that is further developed according to other teachings that can be found in the prior art. In a mutagenesis approach, several exchanges can also be performed simultaneously; that is, inventive and other exchanges can be performed together.

The identity of nucleic acid or amino acid sequences is determined according to the invention through the comparison of sequences. Such a comparison is made by correlating similar sequences in the nucleotide sequences or amino acid sequences to one another. This comparison of sequences is preferably performed on the basis of the BLAST algorithm, which has been established in the prior art and is usually used (for example, see Altschul, S. F., Gish, W., Miller, W., Myers, E. W. & Lipman, D. J. (1990) “Basic local alignment search tool.” J. Mol. Biol. 215:403-410, and Altschul, Stephan F., Thomas L. Madden, Alejandro A. Schaffer, Jinghui Zhang, Hheng Zhang, Webb Miller, and David J. Lipman (1997): “Gapped BLAST and PSI-BLAST: a new generation of Protein database search programs”; Nucleic Acids Res., 25, pp. 3389-3402) and is basically done by correlating similar sequences of nucleotides or amino acids in the nucleic acid or amino acid sequences to one another. A tabular correlation of the respective positions is referred to as alignment. Another algorithm that is available in the prior art is the FASTA algorithm. Sequence comparisons (alignments), particularly multiple sequence comparisons, are usually prepared using computer programs. The Clustal series (for example, see Chenna et al. (2003): Multiple sequence alignment with the Clustal series of programs. Nucleic Acid Research 31, 3497-3500), T-Coffee (for example, see Notredame et al. (2000): T-Coffee: A novel method for multiple sequence alignments. J. Mol. Biol. 302, 205-217) or programs that are based on these programs or algorithms are often used for this purpose. For database searches, Clustal (for example, see Chenna et al. (2003): Multiple sequence alignment with the Clustal series of programs. Nucleic Acid Research 31, 3497-3500), or T-Coffee (for example, see Notredame et al. (2000): T-Coffee: A novel method for multiple sequence alignments. J. Mol. Biol. 302, 205-217) as well as BLAST or FASTA are often used, or programs that are based on these programs or algorithms. In relation to the present invention, sequence comparisons and alignments are preferably prepared using the computer program Vector NTI® Suite 10.3 (lnvitrogen Corporation, 1600 Faraday Avenue, Carlsbad, Calif., USA) using the set default parameters.

Such a comparison provides information regarding the similarity of the compared sequences in relation to one another. This is usually expressed in percent identity, that is, the proportion of identical nucleotides or amino acid residues therein or in an alignment of mutually corresponding positions. The broader concept of homology also takes conserved amino acid exchanges into consideration in the case of amino acid sequences, i.e., amino acids with similar chemical activity, since they usually carry out similar chemical activities within the protein. The similarity of the compared sequences can therefore also be indicated in percent homology or percent similarity. Characterizations can be made about identity and/or homology over entire polypeptides or genes or only about individual regions. Homologous or identical regions of different nucleic acid or amino acid sequences are therefore defined by correspondences in the sequences. Such regions oftentimes have identical functions. They can be small and include only a few nucleotides or amino acids. Such small regions often carry out functions that are essential for the overall activity of the protein. It can therefore be expedient to relate sequence correspondences only to individual, small regions where appropriate. Unless indicated otherwise, however, information on identity or homology provided in the present invention refers to the total length of the respectively indicated nucleic acid or amino acid sequence.

Fragments are understood as being all polypeptides, proteins, or peptides that are smaller than corresponding reference proteins or those which correspond to completely translated genes and can also be obtained synthetically, for example. They can be correlated with the respective complete reference proteins on the basis of their amino acid sequences. For example, they can take on the same spatial structures or carry out proteolytic activities or subactivities, such as the complexation of a substrate.

Fragments and deletion variants of parent proteins are similar in principle; while fragments tend to be smaller parts, the deletion mutants tend to lack only small regions (only one or more amino acids in some circumstances). For instance, it is possible to delete additional individual amino acids at the termini or in the loops of the enzyme without thereby eliminating or diminishing the enzymatic activity.

All indicated quantities for enzymes refer to active protein. The protein concentration can be determined with the aid of known methods, for example the BCA method or the Biuret method. The active protein concentration can be determined in this regard through titration of the active centers using a suitable irreversible inhibitor (for proteases such as phenyl methyl sulfonyl fluoride (PMSF)) and identification of the residual activity (cf. M. Sender et al., J. Am. Chem. Soc. 88, 24 (1966), pp. 5890-5913).

In addition to the amino acid modifications discussed above, enzymes according to the invention can have other amino acid modifications, particularly amino acid substitutions, insertions, or deletions. Such enzymes are further developed and optimized for certain applications or with respect to special characteristics (for example, with respect to their catalytic activity, stability, etc.) by means of targeted genetic modification, i.e., through mutagenesis processes. Furthermore, nucleic acids according to the invention can be used in recombination methods and thus used to produce completely new enzymes or other polypeptides.

The aim is to introduce targeted mutations into the molecules, such as substitutions, insertions, or deletions, in order to improve the cleaning performance of enzymes according to the invention, for example. For this purpose, the surface charges and/or the isoelectric point of the molecules and thus their interactions with the substrate can be changed, in particular. For instance, the net charge of the enzymes can be changed in order to thereby influence the substrate binding particularly for use in detergents and cleaning agents. Alternatively or in addition, one or more appropriate mutations can be used to increase the stability of the amylase, thereby improving its cleaning performance. Advantageous characteristics of individual mutations, e.g., of individual substitutions, can be mutually complementary. An amylase that is already optimized with respect to certain characteristics—for example, with respect to its stability in relation to surfactants/ and/or bleaching agents and/or other components—can therefore also be further developed in the context of the invention.

The following convention is used to describe substitutions that pertain to exactly one amino acid position (amino acid exchanges): First, the amino acid that is naturally present is designated in the form of the internationally used one-letter codes, followed by the associated sequence position, and finally the inserted amino acid. Several exchanges within the same polypeptide chain are separated from one another by forward slashes. In the case of insertions, additional amino acids are named after the sequence position. For deletions, the missing amino acid is replaced by a symbol, such as a star or a hyphen. For example, A95G describes the substitution of alanine at position 95 by glycine, A95AG describes the insertion of glycine after the amino acid alanine at position 95, and A95* describes the deletion of alanine at position 95. This nomenclature is familiar to a person skilled in the field of enzyme technology.

The liquid composition according to the invention must necessarily contain at least one α-amylase that is at least 89% and, in order of increasing preference, at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and up to 100% identical to the sequence indicated in SEQ ID NO.1 over its entire length and, in the count according to SEQ ID NO. 1, has deletions at one or more of positions 180, 181, 182, 183, and 184.

Amino acid positions that are designated in the context of the present invention with the phrasing “count according to SEQ ID NO. 1” are understood as follows: The other amino acid positions are defined by an alignment of the amino acid sequence of an amylase according to the invention with the amino acid sequence as indicated in SEQ ID NO.1. Moreover, the correlation of the positions is based on the mature protein.

This correlation must also be used particularly if the amino acid sequence of a protein according to the invention includes a greater number of amino acid residues than the amylase in SEQ ID NO.1. Starting from the cited positions in the amino acid sequence, the positions of modification in an amylase according to the invention are those which are correlated to precisely these positions in an alignment.

A deletion of two positions selected from among 180+181, 181+182, 182+183, and 183+184 is especially preferred, deletions at positions 183+184 in the count according to SEQ ID NO.1 are very especially preferred, and deletions H183*+G184* are particularly preferred.

The α-amylase of the liquid composition according to the invention in the count according to SEQ ID NO.1 also has an amino acid substitution at one or more of positions 405, 421, 422, and 428. Substitutions I405L; A421 H, A422P, and A428T are especially preferred.

In an especially preferred embodiment, the α-amylase of the liquid composition according to the invention in the count according to SEQ ID NO.1 has deletions H183*+G184* and, additionally, the substitutions I405L, A421 H, A422P, and A428T.

Another object of the present invention is therefore a detergent and cleaning agent containing a combination of an α-amylase and a protease, with the α-amylase being characterized in that it can be obtained from an amylase according to the invention as the parent molecule through single or multiple conservative amino acid substitution. The term “conservative amino acid substitution” refers to the exchange (substitution) of one amino acid residue for another amino acid residue, with this exchange not resulting in a change in the polarity or charge at the position of the exchanged amino acid, e.g., the exchanging of one nonpolar amino acid residue for another nonpolar amino acid residue. In the context of the invention, conservative amino acid substitutions include, for example: G=A=S, l=V=L=M, D=E, N=Q, K=R, Y=F, S=T, G=A=l=V=L=M=Y=F=W=P=S=T.

Another embodiment of the present invention is therefore such a liquid composition, particularly for laundry or for cleaning textiles, containing at least one α-amylase, with the α-amylase being characterized in that it
(a) (i) can be obtained from an α-amylase as the parent molecule through fragmentation, deletion, insertion, or substitution mutagenesis, and
   (ii) said parent molecule is at least 89% and, in order of increasing preference, at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and up to 100% identical to the sequence indicated in SEQ ID NO.1 over its entire length and, in the count according to SEQ ID NO. 1, has deletions at one or more of positions 180, 181, 182, 183, and 184, and
(b) comprises an amino acid sequence that matches with the parent molecule over a length of at least 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 475, 480, 482, 484, or 485 contiguous amino acids.

For instance, it is possible to delete amino acids at the termini or in the loops of the enzyme (parent molecule) without thereby eliminating or diminishing the proteolytic activity. Furthermore, such fragmentation, deletion, insertion, or substitution mutagenesis can also be used to reduce the allergenicity of the enzymes in question, thereby improving their usability as a whole. Advantageously, the enzymes retain their proteolytic activity even after mutagenesis—that is, their proteolytic activity corresponds at least to that of the parent enzyme. Substitutions can also exhibit advantageous effects. Both individual and several contiguous amino acids can be exchanged for other amino acids.

An amylase according to the invention can be additionally stabilized, particularly through one or more mutations, for example substitutions, or through coupling with a polymer. After all, an increase in stability during storage and/or during use, for example during the washing process, has the effect that the enzymatic activity lasts longer, so that the cleaning performance is improved. In principle, all possibilities for stabilization described in the prior art and/or or expedient merit consideration. Such stabilizations are preferred which are achieved through mutations of the enzyme itself, since such stabilizations do not require any additional work steps after the enzyme is obtained. Sequence modifications that are suitable for this purpose are known from the prior art.

Other possibilities for stabilization are, for example:
Changing the bonding of metal ions, particularly of the calcium bonding sites, for example by exchanging one or more of the amino acids involved in the calcium bond for one or more negatively charged amino acids, and/or inserting sequence modifications into at least one of the sequences of the two amino acids arginine/glycine;
Protecting against the influence of denaturing agents such as surfactants through mutation that bring about a modification of the amino acid sequence on or at the surface of the protein;
Exchanging of amino acids that are near the N-terminal for such amino acids that are believed to enter into contact with the rest of the molecule through noncovalent interactions and thus contribute to the maintaining of the globular structure.

Preferred embodiments are those in which the enzyme is stabilized in several ways, since several stabilizing mutations have an additive or synergistic effect.

Another embodiment of the invention is such a liquid composition, particularly for laundry or for cleaning textiles, containing at least one α-amylase as described above that is characterized in that it has at least one chemical modification. An amylase with such a modification is referred to as a derivative, i.e., the amylase is derivatized.

In terms of the present invention, derivatives are therefore to be understood as proteins whose amino acid chain has been chemically modified. Such derivatizations can occur in vivo in the host cell that expresses the protein. Couplings of low-molecular compounds such as lipids or oligosaccharides are particularly noteworthy in this regard. Derivatizations can also be performed in vitro, however, for instance through the chemical conversion of a side chain of an amino acid or through covalent bonding of another compound to the protein. For example, it is possible to couple amines with carboxyl groups of an enzyme in order to change the isoelectric point. This other compound can also be another protein that is bonded to a protein according to the invention via bifunctional chemical compounds, for example. Likewise, derivatization is to be understood as covalent bonding to a macromolecular carrier, or also as noncovalent inclusion in suitable macromolecular cage structures. Derivatizations can influence the substrate specificity or the strength of the bond to the substrate, for example, or bring about a temporary blocking of the enzymatic activity if the coupled substance is an inhibitor. This can be expedient during a period of storage, for example. Such modifications can also influence the stability or the enzymatic activity. Furthermore, they can also be used to reduce the allergenicity and/or immunogenicity of the protein and thereby increase its skin compatibility, for example. For example, coupling with macromolecular compounds, for example polyethylene glycol, can improve the protein in terms of its stability and/or skin compatibility.

Derivatives of a protein according to the invention can also be understood in the broadest sense as preparations of these proteins. Depending on how it is obtained, worked up, or prepared, a protein can be associated with various other substances, for example from the culture of the producing microorganisms. Other substances can have been intentionally added to the protein, for example in order to increase its storage stability.

All preparations of a protein according to the invention are therefore in keeping with the invention. This is also independent of whether or not it actually exhibits this enzymatic activity in a given preparation. After all, it can be desirable for it to have little or no activity during storage and to perform its enzymatic function only at the time of use. This can be controlled by means of appropriate companion substances, for example.

The compositions according to the invention preferably also contain at least one lipase. A lipase contained in a composition according to the invention (particularly a detergent and cleaning agent for textiles that is preferred according to the invention) has lipolytic activity, that it, it is capable of hydrolyzing (lipolyzing) lipids such as glycerides or cholesterol esters. The lipase activity is determined in a manner common in the art, preferably as described in Bruno Stellmach, “Bestimmungsmethoden Enzyme für Pharmazie, Lebensmittelchemie, Technik, Biochemie, Biologie, Medizin” [“Enzyme Determination Methods for Pharmaceutics, Food Chemistry, Engineering, Biochemistry, Biology, Medicine”] (Steinkopff Verlag Darmstadt, 1988, p. 172 et seq.). Lipase-containing samples are added to an olive oil emulsion in emulsifier-containing water and incubated at 30° C. and pH 9.0. Fatty acids are released. These are titrated continuously using an autotitrator for 20 minutes with 0.01 N sodium hydroxide solution, so that the pH value remains constant (“pH-stat titration”). The lipase activity is determined on the basis of the consumption of sodium hydroxide solution with reference to a standard lipase sample. Another suitable method for measuring lipase activity is the release of a dye from a suitable pNP-labeled substrate.

Preferred compositions are characterized in that they contain lipase in a total quantity from 0.01 to 1.0 wt %, particularly from 0.02 to 0.1 wt %, with respect to the total weight of the composition.

Lipase enzymes that are preferred according to the invention are selected from at least one enzyme of the group which comprises triacylglycerol lipase (E.C. 3.1.1.3) and lipoprotein lipase (E.C. 3.1.1.34) and monoglyceride lipase (E.C. 3.1.1.23).

The area of application of the compositions according to the invention that is preferred according to the invention is the cleaning of textiles. Because detergents and cleaning agents for textiles have predominantly alkaline pH values, lipases are used for this purpose in particular, as they are active in the alkaline medium.

Furthermore, the lipase that is preferably contained in a composition according to the invention is present naturally in a microorganism of the type of Thermomyces lanuginosus or Rhizopus oryzae or Mucor javanicus or derived from the previously mentioned naturally present lipases by mutagenesis. Especially preferably, the compositions according to the invention contain at least one lipase that is naturally present in a microorganism of the type of Thermomyces lanuginosus or is derived from the abovementioned lipases that are naturally present in Thermomyces lanuginosus by mutagenesis.

In this context, “naturally present” means that a lipase is an inherent enzyme of the microorganism. Consequently, the lipase can be expressed in the microorganism by a nucleic acid sequence that is part of the chromosomal DNA of the microorganism in its wild-type form. It or the nucleic acid sequence coding for it is therefore present in the wild-type form of the microorganism and/or can be isolated from the wild-type form of the microorganism. In contrast thereto, a lipase or nucleic acid sequence coding for it that is not naturally present in the microorganism would have been introduced in a targeted manner into the microorganism by means of genetic engineering methods, so that the microorganism would have been enriched by the lipase or nucleic acid sequence coding for it. However, a lipase that is naturally present in a microorganism of the type of Thermomyces lanuginosus or Rhizopus oryzae or Mucor javanicus can certainly have been produced recombinantly by another organism.

The fungus Thermomyces lanuginosus (also known as Humicola lanuginosa) belongs to the class of the Eurotiomycetes (subclass Eurotiomycetidae), the order Eurotiales, the family Trichocomaceae, and the genus Thermomyces. The fungus Rhizopus oryzae belongs to the class of the Zygomycetes (subclass lncertae sedis), the order Mucorales, the family Mucoraceae, and the genus Rhizopus. The fungus Mucor javanicus belongs to the class of the Zygomycetes (subclass lncertae sedis), the order Mucorales, the family Mucoraceae, and the genus Mucor. The terms Thermomyces lanuginosus, Rhizopus oryzae, and Mucor javanicus are the biological names of the species within the respective genus.

Lipases that are preferred according to the invention are the lipase enzymes available from Amano Pharmaceuticals under the names Lipase M-AP10®, Lipase LE®, and Lipase F® (also Lipase JV®). Lipase F®, for example, is naturally present in Rhizopus oryzae. Lipase M-AP10, for example, is naturally present in Mucor javanicus.

Compositions of a very especially preferred embodiment of the invention contain at least one lipase that is selected from at least one or more polypeptides with an amino acid sequence that is at least 90% (and, in order of increasing preference, at least 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8%, 99.9%) identical to the wild-type lipase from the strain DSM 4109 Thermomyces lanuginosus. It is also preferred if, starting from said wild-type lipase from the strain DSM 4109, at least the amino acid modification N233R is present.

In the context of another embodiment, particularly those lipases that are derived from the wild-type lipase from the strain DSM 4109 can be preferably used according to the invention and are selected from at least one lipase enzyme according to at least one of claims 1 to 13 of printed publication WO 0060063 A1. Express reference is made to the disclosure of printed publication WO 0060063 A1 in its entirety.

Especially preferably, at least one lipase is used in the compositions of the invention that is derived from the wild-type lipase from the strain DSM 4109 and in which at least one substitution of an electrically neutral or negatively charged amino acid with a positively charged amino acid has occurred. The charge is determined in water at pH 10. Negative amino acids in terms of the invention are E, D, Y, and C. Positively charged amino acids in terms of the invention are R, K, and H, particularly R and K. Neutral amino acids in terms of the invention are G, A, V, L, I, P, F, W, S, T, M, N, Q, and C, if C forms a disulfide bridge.

In the context of this embodiment of the invention, it is also preferred if, starting from the wild-type lipase from the strain DSM 4109, at least one of the following amino acid exchanges is present in positions D96L, T213R and/or N233R, especially preferably T213R and N233R.

It was found that, as a very especially preferred lipase in the compositions according to the invention, at least one lipase is contained which, starting from said wild-type lipase from the strain DSM 4109 Thermomyces lanuginosus, has one of the following amino acid modifications of numbers (L1) to (L41) as the sole modification (modifications in parentheses optional):

| | |
|---|---|
| (L1) | T231R + N233R |
| (L2) | N94K + 096L + T231R + N233R + Q249R + 270P + |
| | 271G + 272L |
| (L3) | 096L + T231R + N233R |
| (L4) | G91A + E99K + T231R + N233R + Q249R |
| (L5) | (N33Q) + 096L + T231R + N233R + Q249R + 270 PGL |
| (L6) | R209P + T231R + N233R |
| (L7) | (N33Q) + E99N + N1018 + T231R + N233R + Q249R + |
| | 270 PGL |
| (L8) | K24C + (N33Q) + 0968 + T231R + N233R + Q249R + |
| | 270 PCL |
| (L9) | (N33Q) + G91A + E99K + T231R + N233R + Q249R + |
| | 270 PGL |
| (L10) | E1A + (N33Q) + G91A + E99K + T231 R + N233R + |
| | Q249R + 270 PGL |
| (L11) | (N33Q) + G91A + E99K + G255R + T231 R + N233R + |
| | Q249R + 270 PGL |
| (L12) | (N33Q) + G91A + E99K + T231 R + N233R + T244R + |
| | Q249R + 270 PGL |
| (L13) | G91A + E99K + T231R + N233R + Q249R |
| (L14) | E87K + G910 + 096L + G225P + T231 R + N233R + |
| | Q249R + N251D |
| (L15) | G91A + E99K + T231R + N233R + Q249R + 270AGVF |
| (L16) | G91A + E99K + T189G + T231R + N233R + Q249R |
| (L17) | 0102G + T231R + N233R + Q249R |
| (L18) | T231R + N233R + Q249R + 270AGVF |
| (L19) | R209P + T231R + N233R |
| (L20) | N33Q + N94K + 096L + T231R + N233R + Q249R + |
| | 270PGLPFKRV |
| (L21) | N33Q + N94K + 096L + T231R + N233R + Q249R |
| (L22) | N33Q + 0968 + T231 R + N233R + Q249R |
| (L23) | N33Q + 0968 + V2281 + T231R + N233R + Q249R |
| (L24) | E1A + N33Q + G91A + E99K + T231R + N233R + |
| | Q249R + 270PGLPFKRV |
| (L25) | N33Q + 883T + E87K + G91A + E99K + T231R + |
| | N233R + Q249R |
| (L26) | N33Q + G91A + E99K + T231R + N233R + Q249R + |
| | 270PGLPFKRV |
| (L27) | T231R + N233R + 270CP |
| (L28) | T231R + N233R + 270RE |
| (L29) | N33Q + E99N + N1018 + T231R + N233R + Q249R + |
| | 270PGLPFKRV |
| (L30) | 062A + 883T + G91A + E99K + T231R + N233R + |
| | Q249R |
| (L31) | E99N + N1018 + T231R + N233R + Q249R |
| (L32) | R84W + G91A + E99K + T231R + N233R + Q249R |
| (L33) | G91A + E99K + T231R + N233R + Q249R + 2708PG |
| (L34) | G91A + E99K + T231R + N233R + Q249R + 270VVVP |
| (L35) | G91A + E99K + T231R + N233R + Q249R + |
| | 270LLA88GRGGHR |
| (L36) | G91 + E99K + T231R + N233R + Q249R + 270VTT |
| (L37) | G91A + E99K + T231R + N233R + Q249R + 270VLQ |
| (L38) | G91A + E99K + T231R + N233R + Q249R + 270T8T |
| (L39) | G91A + E99K + T231R + N233R + Q249R + 270LRI |
| (L40) | V60G + D62E + G91A + E99K + T231R + N233R + |
| | Q249R |
| (L41) | G91A + D96W + E99K + T231R + N233R + G263Q + |
| | L264A + 1265T + G2668 + T267A + L269N + 270AGGF8 |

Preferred compositions of this embodiment are characterized in that they contain said polypeptide in a total quantity from 0.01 to 1.0 wt %, particularly from 0.02 to 0.1 wt %, with respect to the total weight of the composition.

One highly preferred lipase is available commercially under the trade name Lipex® from Novozymes (Denmark) and can be used advantageously in the cleaning compositions according to the invention. Lipase Lipex® 100 L (from Novozymes A/S, Denmark) is especially preferred in this regard. Preferred compositions are characterized in that they contain said lipase enzyme from Lipex® 100 L in a total quantity from 0.01 to 1.0 wt %, particularly from 0.02 to 0.1 wt %, with respect to the total weight of the composition.

The compositions according to the invention preferably also contain at least one mannanase. In the context of its mannanase activity, a mannanase contained in the composition according to the invention (particularly in a detergent and cleaning agent for textiles that is preferred according to the invention) catalyzes the hydrolysis of 1,4-beta-D-mannosidic bonds in mannans, galactomannans, glucomannans, and galactoglucomannans. These mannanase enzymes according to the invention are classified according to the enzyme nomenclature as E.C. 3.2.1.78.

The mannanase activity of a polypeptide or enzyme can be determined according to test methods known from the literature. For example, an assay solution is introduced into holes of an agar plate with a 4 mm diameter containing 0.2 wt % AZGL galactomannan (carob), i.e., substrate for the endo-1,4-beta-D-mannanase assay, available under catalog number 1-AZGMA from Megazyme (http://www.megazyme.com).

Suitable compositions according to the invention contain the mannanase that is marketed by Novozymes under the name Mannaway®, for example.

Mannanase enzymes have been identified in numerous Bacillus organisms:

WO 9964619 discloses examples of liquid, protease-containing detergent compositions having a high total surfactant content of at least 20 wt % which additionally comprise mannanase enzyme.

Preferably, the compositions according to the invention are characterized in that they contain mannanase in a total quantity from 0.01 to 1.0 wt %, particularly from 0.02 to 0.1 wt %, with respect to the total weight of the composition.

Mannanase polypeptides from strains of the Thermoanaerobacter group, such as Caldicellulosiruptor, are especially suitable according to the invention. Mannanase polypeptides of the fungi Humicola or Scytalidium, particularly of the species Humicola insolens or Scytalidium thermophilum, can also be used in the context of the invention.

It is especially preferred according to the invention if the compositions according to the invention contain, as a mannanase enzyme, at least one mannanase polypeptide from Gram-positive alkalophilic strains of Bacillus, particularly selected from at least one representative of the group of Bacillus subtilis, Bacillus lentus, Bacillus clausii, Bacillus agaradhaerens, Bacillus brevis, Bacillus stearothermophilus, Bacillus alkalophilus, Bacillus amyloliquefaciens, Bacillus coagulans, Bacillus circulans, Bacillus lautus, Bacillus thuringiensis, Bacillus cheniformis, and Bacillus sp., especially preferably selected from at least one representative of the group of Bacillus sp. 1633, Bacillus sp. AAI12, Bacillus clausii, Bacillus agaradhaerens and Bacillus licheniformis.

A preferred mannanase according to the invention is selected from at least one representative of the group that is formed by
i) polypeptides comprising an amino acid sequence that has at least 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7% or 99.8%) sequence identity to the polypeptide according to SEQ ID No.1 (cf. sequence listing), and
ii) polypeptides that are a fragment of (i).

It is preferred, in turn, if the preferred mannanase is contained in the composition according to the invention in a total quantity from 0.01 to 1.0 wt %, particularly from 0.02 to 0.1 wt %, each with respect to the total weight of the composition.

A preferred mannanase enzyme is disclosed according to claim 1 of WO 9964619 and described in greater detail in the description of this WO printed publication and, accordingly, is selected from among at least one mannanase enzyme that is selected from at least one representative of the group that is made up of
i) polypeptides that can be coded by the portion of the DNA sequence coding for the mannanase enzyme that is cloned into the plasmid present in Escherichia coli DSM 12197,
ii) polypeptides that comprise an amino acid sequence as shown at positions 33-340 of SEQ ID NO.2 in WO 9964619,
iii) polypeptides that comprise an amino acid sequence as shown at positions 31-990 or positions 91-1470, each of SEQ ID NO.2 in WO 9964619, or
iv) analogs of the polypeptides defined in (i) or (ii) that have at least 90% (and, in order of increasing preference, at least 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8%, 99.9%) sequence identity with the polypeptide or are a fragment of (i), (ii), or (iii).

For the interpretation of the features of the above-defined mannanase enzymes preferred according to the invention (vide supra), the entire disclosure of WO 9964619 must also expressly be used. The mannanase enzymes cited as being preferred in WO 9964619 are likewise preferred in terms of the composition according to the invention.

Compositions that are preferred according to the invention additionally contain at least one protease. A protease is an enzyme that cleaves peptide bonds by means of hydrolysis. Each of the enzymes from class E.C. 3.4 is included among those according to the invention (including any of the thirteen subclasses thereof). The EC number corresponds to the 1992 enzyme nomenclature of NC-IUBMB, Academic Press, San Diego, Calif., including supplements 1 to 5, published in Eur. J. Biochem. 1994, 223, 1-5; Eur. J. Biochem. 1995, 232, 1-6; Eur. J. Biochem. 1996, 237, 1-5; Eur. J. Biochem. 1997, 250, 1-6; and Eur. J. Biochem. 1999, 264, 610-650.

Subtilase refers to a subgroup of the serine proteases. The serine proteases or serine peptidases are a subgroup of the proteases that possess serine in the active center of the enzyme that forms a covalent adduct with the substrate. Furthermore, the subtilases (and the serine proteases) are characterized in that, besides the aforementioned serine with histidine and aspartame, they have two additional amino acid residues in the active center. The subtilases can be divided into 6 subclasses, namely the subtilisin family, the thermitase family, the proteinase K family, the family of the antibiotic peptidases, the kexin family, and the pyrolysine family. Proteases that are preferably excluded as a component of the compositions according to the invention or contained in reduced quantities are endopeptidases (E.C. 3.4.21).

“Protease activity” exists according to the invention if the enzyme has proteolytic activity (EC 3.4). Various protease activity types are known: The three main types are:

Trypsin-like, in which a cleavage of the amide substrate occurs after the amino acids Arg or Lys at P1; chymotrypsin-like, in which a cleavage occurs after one of the hydrophobic amino acids at P1; and elastase-like, in which a cleavage of the amide substrate occurs after Ala at P1.

The protease activity can be determined using the method described in Tenside [Surfactants], Volume 7 (1970), pp. 125-132. It is indicated accordingly in PU (protease units). The protease activity of an enzyme can be determined using established standard methods, such as, in particular, through the use of BSA as a substrate (bovine albumin) and/or using the AAPF method.

It is preferred according to the invention if the liquid compositions additionally contain at least one cellulase. A cellulase is an enzyme. Synonymous terms can be used for cellulases, such as endoglucanase, endo-1,4-beta glucanase, carboxymethyl cellulase, endo-1,4-beta-D-glucanase, beta-1,4-glucanase, beta-1,4-endoglucan hydrolase, celludextrinase, or avicelase. The crucial factor for determining whether an enzyme is a cellulase in terms of the invention is its ability to hydrolyze 1,4-β-D-glucosidic bonds in cellulose.

Cellulases (endoglucanases, EG) that can be manufactured according to the invention include, for example, the fungal, endoglucanase (EG)-rich cellulase preparations or further developments thereof that are offered by Novozymes under the trade name Celluzyme®. The products Endolase® and Carezyme®, which are also available from Novozymes, are based on the 50 kD EG and 43 kD EG, respectively, from Humicola insolens DSM 1800. Other commercial products from this company that can be used are Cellusoft®, Renozyme®, and Celluclean®. Examples of others that can be used are cellulases that are available from AB Enzymes, Finland, under the trade names Ecostone® and Biotouch® and that based at least in part on the 20 kD EG from Melanocarpus. Other cellulases from AB Enzymes are Econase® and Ecopulp®. Other suitable cellulases are from Bacillus sp. CBS 670.93 and CBS 669.93, with the cellulase from Bacillus sp. CBS 670.93 being available from Danisco/Genencor under the trade name Puradax®. Other commercial products from Danisco/Genencor that can be used are “Genencor detergent cellulase L” and lndiAge®Neutra.

Variants of these enzymes that can be obtained through point mutation can also be used according to the invention. Especially preferred cellulases are Thielavia terrestris cellulose variants that are disclosed in international published patent application WO 9812307, cellulases from Melanocarpus, particularly Melanocarpus albomyces, that are disclosed in international published patent application WO 9714804, cellulases of the EGlll type from Trichoderma reesei that are disclosed in European patent application EP 1305432 and variants thereof, particularly those disclosed in European patent applications EP 1240525 and EP 1305432, as well as cellulases that are disclosed in international published patent applications WO 1992006165, WO 9629397, and WO 02099091. Express reference is therefore made to the respective disclosure and the relevant disclosed content expressly incorporated into the present patent application.

Compositions that are especially preferred according to the invention are characterized in that they contain, as an additional cellulase, at least one 20K cellulase that can be obtained from Melanocarpus sp. or Myriococcum sp. or one having a homology thereto of greater than 80% (in order of increasing preference, greater than 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8%, 99.9%).

The 20K cellulase that can be obtained from Melanocarpus sp. oder Myriococcum sp. is known from international patent application WO 9714804. As described therein, it has a molecular weight of about 20 kDa and, at 50° C. in the pH range from 4 to 9, has at least 80% of its maximum activity, with almost 50% of the maximum activity still being maintained at pH 10. As also described therein, it can be isolated from Melanocarpus albomyces and produced in Trichoderma reseei transformants manufactured by genetic engineering. Cellulases having a homology of greater than 80% (increasingly preferably of greater than 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8%, 99.9%) to the 20K cellulase are also usable in terms of the present invention.

K20 cellulases are preferably used in such quantities that a composition according to the invention has a cellulolytic activity from 1 NCU/g to 500 NCU/g (determinable through the hydrolysis of 1 wt % carboxymethyl cellulose at 50° C. and neutral pH and the determination of the reducing sugar released using dinitrosalicylic acid, as described by M. J. Bailey et al. in Enzyme Microb. Technol. 3: 153 (1981); 1 NCU defines the quantity of enzyme that produces reducing sugar in an amount that corresponds to 1 nmol glucose per second), particularly from 2 NCU/g to 400 NCU/g, and especially preferably from 6 NCU/g to 200 NCU/g. In addition, the composition according to the invention can optionally contain other cellulases.

A composition according to the invention preferably contains 0.001 mg to 0.5 mg, particularly 0.02 mg to 0.3 mg of cellulolytic protein per gram of the overall composition. The protein concentration can be determined with the aid of known methods, such as the bicinchoninic acid method (BCA-Verfahren, Pierce Chemical Co., Rockford, Ill.) or the Biuret method (A. G. Gornall, C. S. Bardawill and M. M. David, J. Biol. Chem. 177, 751-766, 1948).

It is also especially preferred according to the invention to use, in addition to at least one first 20K cellulase that can be obtained from Melanocarpus sp. or Myriococcum sp. or having a homology thereto of greater than 80% (in order of increasing preference, greater than 81%, 82%, 83%, 84%, 85%, 86%, 87%, 88%, 89%, 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99.0%, 99.1%, 99.2%, 99.3%, 99.4%, 99.5%, 99.6%, 99.7%, 99.8%, 99.9%), at least one additional second cellulase that is different from the first cellulase.

In general, the enzymes contained in a composition according to the invention can be adsorbed on carriers and/or embedded in coating substances in order to protect them from premature inactivation.

The enzymes obtained can be added to compositions according to the invention in any form established according to the prior art. These include, particularly, the solid preparations obtained through granulation, extrusion, or lyophilization, advantageously concentrated to the greatest possible extent, with low-moisture, and/or with stabilizers added. In an alternative pharmaceutical form, the enzymes can also be encapsulated, for example through spray-drying or extrusion of the enzyme solution together with a preferably natural polymer or in the form of capsules, for example those in which the enzymes are enclosed in a set gel, or in those of the core-shell type in which an enzyme-containing core is coated with a water-, air-, and/or chemical-impermeable protective layer. In the case of overlaid layers, other active substances, such as stabilizers, emulsifiers, pigments, bleaching agents, or dyes, can be additionally applied. Such capsules are applied using inherently known methods, for example through shaking or roll granulation or in fluidized bed processes. Such granulates are advantageously low in dust, for example due to the application of polymeric film-formers, and stable in storage due to the coating.

At 25° C. and 1013 mbar, the composition according to the invention necessarily has a water activity a_{w} between 0.3 and 0.95, preferably in a range from 0.4 to 0.95.

It is especially preferred according to the invention if, at 25° C. and 1013 mbar the liquid composition has a water activity a_{w} of no more than 0.9, particularly no more than 0.8, very especially preferably no more than 0.7, most preferably no more than 0.6. For each of abovementioned preferred maximum, the liquid composition must, at 25° C. and 1013 mbar, have a water activity a_{w} of greater than 0.3, preferably of at least 0.4.

Particularly if the liquid composition according to the invention is to be presented as a portion enclosed in a water-soluble film, the composition according to the invention, at 25° C. and 1013 mbar, preferably has a water activity a_{w} between 0.3 and 0.6, preferably in a range from 0.35 to 0.55. It is very especially preferred if these compositions contain as surfactant a combination of surfactants designated as being preferred (vide supra), particularly one of the aforementioned combinations of surfactants (A) to (D).

The water activity a_{w} is a number from 0 to 1. It is a measure of the free water contained in the composition. The water activity can be determined by measuring the air humidity over the sample in a sealed sample chamber at 25° C. and 1013 mbar. The humidity of the air above the sample in the equilibrium state is determined in % RH (relative humidity in %). % RH and temperature are measured until an equilibrium is reached for % RH and temperature (at 25° C.).

The value a_{w} is in linear relation to the measured relative humidity % RH (1.00 a_{w}=100% RH).

The measuring device used to determine the a_{w} value is calibrated using standardized, saturated salt solutions according to the National Bureau of Standards (NBS), Washington D.C., (Journal of Research of the National Bureau of Standards, Volume 81A, No. 1, January/February 1977) and according to the Physikalisch-Technische Bundesanstalt, Berlin (test report no. PTB-2.54-1063/-4180/93, August 1993).

The water activity a_{w} was determined using the LabMaster-a_{w} measuring instrument (sensor cell type: CM-2) from Novasina (Switzerland). The standardized salt solutions SAL-T 6 (6% RH), SAL-T 11 (11% RH), SAL-T 33 (33% RH), SAL-T 58 (58% RH), SAL-T 75 (75% RH), SAL-T 84 (84% RH), and SAL-T 97 (97% RH) (all from Novasina) were each used to calibrate the measurement of the relative atmospheric humidity according to the abovementioned standards. The calibration of the device was performed and certified by Novasina in accordance with the abovementioned standards.

To determine the a_{w} value, the substance to be measured (7.5 mL) was filled into a sample vessel of the aforementioned measuring instrument, the sample vessel was placed immediately into the measuring chamber, and the measuring chamber was closed. The measurement temperature in the measuring chamber was maintained at 25° C. If the a_{w} value remained stable for 8 minutes (±0.001 a_{w}) (device setting a_{w} factor: 8 minutes) and the temperature remained stable for 4 minutes (25° C.±0.1° C.) (device setting temperature factor: 4 minutes), the equilibrium state had been reached, and the measured value was outputted as a_{w}.

In addition to the required ingredients, the compositions according to the invention can contain other ingredients that further improve the technical and/or aesthetic characteristics of the detergent. In the context of the present invention, the composition according to the invention preferably also contains one or more substances from the group of the bleaching agents, complexing agents, builders, electrolytes, pH adjusters, perfumes, perfume carriers, fluorescent agents, dyes, hydrotropes, foam inhibitors, silicone oils, antiredeposition agents, shrinkage inhibitors, anti-creasing agents, color transfer inhibitors, antimicrobial agents, germicides, fungicides, antioxidants, preservatives, corrosion inhibitors, antistatic agents, bittering agents, ironing aids, repellents and impregnating agents, swelling and antislip agents, softening components, and UV absorbers.

Any substances can be used as bleaching agents which destroy and/or take up dyes through oxidation, reduction, or adsorption, thereby decolorizing materials. These include inter alia hypohalogenite-containing bleaching agents, hydrogen peroxide, perborate, percarbonate, peracetic acid, diperazelaic acid, diperoxydodecanedioic acid, and oxidative enzyme systems.

Some noteworthy builders that can be contained in the composition according to the invention are particularly silicates, aluminum silicates (particularly zeolites), carbonates, salts of organic di- and polycarboxylic acids, as well as mixtures of these substances.

Organic builders that can be present in the composition according to the invention are the polycarboxylic acids, for example, which can be used in the form of their sodium salts, with polycarboxylic acids being understood as being such carboxylic acids that have more than one acid function. For example, these are citric acid, adipic acid, succinic acid, glutaric acid, malic acid, tartaric acid, maleic acid, fumaric acid, sugar acids, aminopolycarboxylic acids, as well as mixtures thereof. Preferred salts are the salts of the polycarboxylic acids such as citric acid, adipic acid, succinic acid, glutaric acid, tartaric acid, sugar acids, and mixtures thereof.

Polymeric polycarboxylates are also suitable as builders. These are the alkali metal salts of polyacrylic acid or of polymethacrylic acid, for example, such as those having a relative molecular mass from 600 to 750,000 g/mol.

Suitable polymers are particularly polyacrylates, which preferably have a molecular mass from 1,000 to 15,000 g/mol. Due to their superior solubility, the short-chain polyacrylates, which have molar masses from 1,000 to 10,000 g/mol, and especially preferably from 1,000 to 5,000 g/mol, can be preferred from this group.

Copolymeric polycarboxylates, particularly those of acrylic acid with methacrylic acid, and acrylic acid or methacrylic acid with maleic acid, are also suitable. To improve water solubility, the polymers can also contain allyl sulfonic acids such as allyloxybenzene sulfonic acid and methallyl sulfonic acid as monomers.

In the liquid compositions according to the invention, soluble builders such as citric acid, for example, or acryl polymers having a molar mass from 1,000 to 5,000 g/mol are preferably used.

A second object of the invention is the use of a liquid composition according to the first object of the invention to stabilize α-amylase that is at least 89% and, in order of increasing preference, at least 90%, 90.5%, 91%, 91.5%, 92%, 92.5%, 93%, 93.5%, 94%, 94.5%, 95%, 95.5%, 96%, 96.5%, 97%, 97.5%, 98%, 98.5%, 99%, 99.5% and up to 100% identical to the sequence indicated in SEQ ID NO.1 over its entire length and, in the count according to SEQ ID NO. 1, has deletions at one or more of positions 180, 181, 182, 183, and 184.

A third object of the invention is the use of a liquid composition according to the first object of the invention to clean textiles.

A fourth object of the invention is a method for cleaning textiles, comprising the provision of a washing liquor using at least the following components:
(i) at least one composition according to the first object of the invention,
(ii) preferably at least one solvent, particularly water, and
(iii) at least one textile.

According to the invention, a washing liquor is at least the total quantity of the components enumerated under (i) and (iii).

Methods for cleaning textiles are generally characterized in that various substances that are active in detergency are applied to the article to be cleaned in several method steps and washed off after the treatment time, or that the article to be cleaned is otherwise treated with a composition according to the first object of the invention or a solution of this composition.

If component (ii) of the method according to the invention is additionally added to the washing liquor, then it is preferred according to the invention to combine one part by volume of component (i) with 5 to 3000 parts by volume of component (ii).

In the described methods, temperatures of 60° C. or below, 40° C. or below, 30° C. or below, or 20° C. or below are used in various embodiments of the invention. These temperatures refer to the temperatures used in the washing steps.

### EXAMPLES

The following compositions were prepared:

1. Liquid Detergent:

| | V1 | E1 |
|---|---|---|
| | Quantity | Quantity |
| Ingredient | in wt % | in wt % |
| Boric acid | 1.0 | 1.0 |
| Citric acid monohydrate | 2.2 | 3.2 |
| Fatty alcohol ether sulfate with 2 mol EO | 7.0 | 9.0 |
| Fatty alcohol ether with 7 EO | 5.5 | 9.0 |
| Alkylbenzene sulfonic acid | 5.5 | 7.0 |
| NaOH | 2.33 | 2.95 |
| Glycerin | 2.5 | — |
| Diethylenetriamine penta(methylene | 0.3 | 0.3 |
| phosphonic acid), heptasodium salt | | |
| Fatty acid soap | 2.2 | 4.0 |
| Ethanol | 1.2 | 2.0 |
| 1,2-propanediol | — | 5.5 |
| Amylase (according to the invention) | 0.44 | 0.44 |
| Mannanase | 0.10 | 0.10 |
| Lipase | 0.20 | 0.20 |
| Cellulase | 0.15 | 0.15 |
| Protease | 0.65 | 0.65 |
| Pectatlyase | 0.05 | 0.05 |
| Water | Up to 100 | Up to 100 |
| Water activity a_{w} | 0.956 | 0.928 |

The water activity was determined (for measurement report, vide supra).

After storage for 4 weeks at 30° C., composition E1 according to the invention had greater amylase activity than reference composition V1.

| | | Amylase activity | Amylase activity |
|---|---|---|---|
| | | after storage for 2 | after storage for 4 |
| Formula | Initial value | weeks at 40° C. | weeks at 40° C. |
| E1 | 100% | 90% | 60% |
| V1 | 100% | 71% | 47% |

Determination of Washing Performance in Washing Test

In a washing machine of type Miele Softtronic W 1734, standardized white test laundry (3.5 kg) that had been freshly soiled in a standardized manner was washed using the “easy care” washing program for a time period of 70 minutes and a water level of 17 liters (hardness 16 d) at 30° C.

Composition E1 according to the invention had the best washing performance.

2.0 Detergent Pouch with Solid and Liquid Composition

The following compositions were prepared and packed as described below in a water-soluble film of polyvinyl alcohol:

| | L1 |
|---|---|
| Liquid composition | [wt %] |
| C₁₁₋₁₃ alkylbenzene sulfonic acid | 23.0 |
| C₁₃₋₁₅ alkyl alcohol ethoxylated with 8 mol ethylene | 24.0 |
| oxide | |
| Glycerin | 9.0 |
| 2-amino ethanol | 6.8 |
| Ethoxylated polyethylenimine | 4.0 |
| C₁₂-C₁₈ fatty acid | 7.5 |
| Diethylene triamine-N,N,N′,N′,N″- | 3.5 |
| penta(methylene phosphonic acid), heptasodium | |
| salt | |
| 1,2 propylene glycol | 4.5 |
| Ethanol | 4.0 |
| Soil-release polymer of ethylene terephthalate and | 1.0 |
| polyethylene oxide terephthalate | |
| Amylase (according to the invention) | 0.6 |
| Perfume, dye | 1.7 |
| Water | Up to 100 |
| Water activity a_{w} | 0.42 |

| | F1 |
|---|---|
| Solid composition (granulate) | [wt %] |
| Sodium percarbonate (granulate) | 35.0 |
| TAED | 13.0 |
| Carboxymethyl cellulose | 4.0 |
| Sodium hydrogen carbonate | 9.0 |
| Sodium silicate (SiO₂)_{2.5}(Na₂O) | 11.0 |
| C₁₈ fatty acid soap | 2.6 |
| Enzyme (protease, lipase, mannanase) in the | 10.7 |
| granulate of sodium sulfate | |
| Sodium sulfate | Up to 100 |

An M8613 film from Monosol (88 μm) was stretched onto a mold having a dual cavity. The stretched film is heated for a duration of 2400 ms at 105° C. by contact heating and then suctioned by a vacuum into the cavity. 8.5 g of the solid composition F1 is then preweighed and filled into the first cavity, and then 16.5 of liquid composition L1 is placed into the second cavity using a syringe. A top film (M8630, 90 μm) was then put in place in order to seal the cavities and welded with the first film using heat (150° C., 1000 ms). After breaking the vacuum, the portion was removed from the cavity. A wall of the powder chamber for the portion was then perforated with a needle.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A liquid composition, particularly for cleaning textiles, containing
(a) at least one surfactant, and
(b) at least one α-amylase that is at least 89% identical to the sequence indicated in SEQ ID NO.1 over its entire length and, in the count according to SEQ ID NO. 1, has deletions at one or more of positions 180, 181, 182, 183, and 184,
with the proviso that, at 25° C. and 1013 mbar, the composition has a water activity a_{w} between 0.3 and 0.95.

2. The liquid composition as set forth in claim 1, characterized in that, at 25° C. and 1013 mbar, the composition has a water activity a_{w} of no more than 0.9.

3. The liquid composition as set forth in claim 2, characterized in that, at 25° C. and 1013 mbar, the composition has a water activity a_{w} of at least 0.4.

4. The liquid composition as set forth in claim 1, characterized in that, with respect to the total weight of the composition, the at least one surfactant is contained in a total quantity from 5.0 to 60.0 wt %.

5. The liquid composition as set forth in claim 1, characterized in that the at least one surfactant is an anionic surfactant having a structure of formula (A-3) 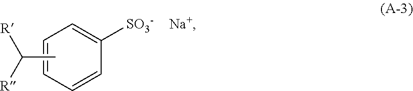
wherein R, R″ together contain 9 to 19 C atoms.

6. The liquid composition as set forth in claim 1, characterized in that the at least one surfactant is at least one fatty alcohol ether sulfate of formula (A-1) 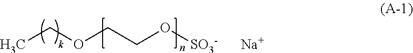
wherein k=11 to 19, n=2, 3, 4, 5, 6, 7, or 8.

7. The liquid composition as set forth in claim 1, characterized in that the at least one surfactant comprises at least one nonionic surfactant.

8. The liquid composition as set forth in claim 1, characterized in that the at least one α-amylase has deletions in at least two positions selected from among positions 180+181, 181+182, 182+183, and 183+184 in the count according to SEQ ID NO.1.

9. The liquid composition as set forth in claim 1, characterized in that the at least one α-amylase has deletions H183+G184 in the count according to SEQ ID NO.1.

10. The liquid composition as set forth in claim 1, characterized in that, in the count according to SEQ ID NO.1, the at least one α-amylase has amino acid substitutions at one or more of positions 405, 421, 422, and 428.

11. The liquid composition as set forth in claim 1, characterized in that, in the count according to SEQ ID NO.1, the at least one α-amylase substitutions I405L, A421 H, A422P, and A428T.

12. The liquid composition as set forth in claim 1, further comprising at least one protease.

13. The liquid composition as set forth in claim 12, characterized in that the at least one lipase is selected from one or more polypeptides having an amino acid sequence that is at least 90% identical to the wild-type lipase from the strain DSM 4109 Thermomyces lanuginosus.

14. The liquid composition as set forth in claim 13, characterized in that the at least one lipase is selected from one or more polypeptides having an amino acid sequence derived from the wild-type lipase from strain DSM 4109 in which at least one of the following amino acid exchanges is present in positions D96L, T213R, and/or N233R.


SEQUENCE LISTING (1 sequence)

SEQ ID NO: 1 - protein, 485 aa, Artificial
  Hybridprotein
HHNGTNGTMMQYFEWYLPNDGNHWNRLNSDASNLKSKGITAVWIPPAWKGASQNDVGYGAYDLYDLGEFNQKGTVRTKYGTRSQLQAAVTSLKNNGIQVYGDVVMNHKGGADATEMVRAVEVNPNNRNQEVTGEYTIEAWTRFDFPGRGNTHSSFKWRWYHFDGVDWDQSRRLNNRIYKFRGHGKAWDWEVDTENGNYDYLMYADIDMDHPEVVNELRNWGVWYTNTLGLDGFRIDAVKHIKYSFTRDWINHVRSATGKNMFAVAEFWKNDLGAIENYLQKTNWNHSVFDVPLHYNLYNASKSGGNYDMRNIFNGTVVQRHPSHAVTFVDNHDSQPEEALESFVEEWFKPLAYALTLTREQGYPSVFYGDYYGIPTHGVPAMRSKIDPILEARQKYAYGPQHDYIDHPDVIGWTREGDSSAAKSGLAALITDGPGGSKRMYAGLKNAGETWYDITGNRSDTVKIGSDGWGEFHVNDGSVSIYVQK